(19) United States
(12) Patent Application Publication
(10) Pub. No.: US 2006/0094906 A1
(43) Pub. Date: May 4, 2006

(54) Catalyst for partial oxidation of methylbenzenes and method for producing aromatic aldehydes using the same

(76) Inventors: Won-ho Lee, Seo-gu (KR); Dong-il Lee, Dongducheon-si (KR); Jong-hyun Chae, Yuseong-gu (KR); Hyun-kyung Yoon, Seoul (KR)

Correspondence Address: MCKENNA LONG & ALDRIDGE LLP, 1900 K STREET, NW, WASHINGTON, DC, 20006, US

(21) Appl. No.: 11/254,765

(22) Filed: Oct. 21, 2005

(30) Foreign Application Priority Data
Nov. 4, 2004 (KR) 10-2004-0089376

(51) Int. Cl. C07C 45/36

(52) U.S. Cl. 568/431

(57) ABSTRACT

Provided is a catalyst for partial oxidation of methylbenzenes comprising the compound represented by the following formula 1 and, optionally, a fire-resistant inorganic support:
WOₓ (1) where W stands for a tungsten atom, O stands for an oxygen atom and x is a number determined by the oxidation state of W. Also provided is a method for producing aromatic aldehydes from partial oxidation of methylbenzenes in gas phase using molecular oxygen using the afore-mentioned catalyst. The catalyst of the present invention can be prepared easily compared with conventional multi-component oxide catalysts. And, aromatic aldehydes can be produced from methylbenzenes with high selectivity and yield.

This application claims the benefit of the filing date of Korean Patent Application No. 10-2004-0089376 filed on Nov. 4, 2004, in the Korean Intellectual Property Office, the disclosure of which is incorporated herein in its entirety by reference.

### TECHNICAL FIELD

The present invention relates to a catalyst for partial oxidation of methylbenzenes and a method for producing aromatic aldehydes using the same. More particularly, the invention relates to a catalyst adequate for producing aromatic aldehydes in high yield from partial oxidation of methylbenzenes in gas phase using molecular oxygen and a method for producing aromatic aldehydes in high yield from partial oxidation of methylbenzenes in gas phase with molecular oxygen using the catalyst.

### BACKGROUND ART

Since aromatic aldehydes have highly reactive aldehyde groups, they can be used for a variety of purposes. Especially, terephthalaldehyde, which has two aldehyde groups in the para positions, is drawing attention for use as basic material in the field of medicines, agrichemicals, pigments, liquid crystal polymers, conducting polymers, heat resistance plastics, etc.

For conventional methods for producing terephthalaldehyde, there are dehydration of chlorinated p-xylene intermediate, hydrogenation of dimethyl terephthalate, etc. These methods are inadequate for mass production of terephthalaldehyde because of complicated process, high-pressure and environment-unfriendly condition, etc.

Efforts have been made to overcome these problems and enable mass production of terephthalaldehyde by oxidation of p-xylene in gas phase using molecular oxygen. Japanese Patent Laid-Open No. Sho 47-002086 disclosed a mixed oxide catalyst comprising W and Mo in the range from 1:1 to 20:1. Japanese Patent Laid-Open No. Sho 48-047830 disclosed a catalyst comprising V and Rb or Cs. U.S. Pat. No. 3,845,137 disclosed a catalyst comprising W, Mo and at least one element selected from a group consisting of Ca, Ba, Ti, Zr, Hf, Tl, Nb, Zn and Sn. U.S. Pat. No. 4,017,547 disclosed a catalyst comprising an oxide of Mo, an oxide of W or silicotungstic acid and an oxide of Bi. However, these catalysts are limited in industrial use because of low terephthalaldehyde selectivity and yield.

U.S. Pat. No. 5,324,702 disclosed a catalyst in which at least one element selected from a group consisting of Fe, Zn, Zr, Nb, In, Sn, Sb, Ce and Bi and at least one element selected from a group consisting of V, Mo and W are supported on a deboronized borosilicate crystal molecular sieve by chemical vapor deposition (CVD). Although this catalyst shows relatively higher p-xylene conversion rate and terephthalaldehyde yield than the conventional catalysts, selectivity improvement and separation and purification are difficult because of a variety of byproducts.

Recently, U.S. Pat. No. 6,458,737 B1 disclosed a catalyst comprising W, as main constituent, and at least one element selected from a group consisting of Sb, Fe, Co, Ni, Mn, Re, Cr, V, Nb, Ti, Zr, Zn, Cd, Y, La, Ce, B, Al, Tl, Sn, Mg, Ca, Sr, Ba, Li, Na, K, Rb and Cs. This catalyst shows high terephthalaldehyde yield enabling industrial use. However, in spite of high p-xylene conversion rate, terephthalaldehyde selectivity is not so high and the Sb component tends to be lost at high temperature due to sublimation. Thus, the catalyst has problems in thermal stability and catalyst life.

To summarize, conventional catalysts are limited in industrial use because separation and purification are difficult due to low terephthalaldehyde yield or terephthalaldehyde selectivity and because the catalysts tend to have non-uniform composition and capacity due to use of multi-component oxides. Besides, since they comprise the components having poor thermal stability, they tend to have short life.

### DISCLOSURE OF INVENTION

It is an object of the present invention to provide a catalyst for partial oxidation of methylbenzenes enabling production of aromatic aldehydes from methylbenzenes with good selectivity and high yield and having uniform composition and capacity and a method for producing aromatic aldehydes from methylbenzenes with good selectivity and high yield using the same.

To attain the object, the present invention provides a catalyst for partial oxidation of methylbenzenes comprising the compound represented by the following formula 1:
WOₓ (1)

where W stands for a tungsten atom, O stands for an oxygen atom and x is a number determined by the oxidation state of W, preferably in the range of 2 to 3.

The catalyst of the present invention can be supported on a fire-resistant inorganic support.

The present invention also provides a method for producing aromatic aldehydes by partial oxidation of methylbenzenes in gas phase using molecular oxygen using the compound represented by the formula 1, alone or as supported on a fire-resistant inorganic support, as catalyst.

Hereunder is given a detailed description of the invention.

Methylbenzenes refer to the compounds wherein at least one methyl group is directly bonded to the benzene ring. Typical examples are those having 8 to 10 carbon atoms, such as p-xylene, o-xylene, m-xylene, pseudocumene, mesitylene and durene.

The catalyst of the present invention is for producing aromatic aldehydes from partial oxidation of methylbenzenes in gas phase using molecular oxygen. For example, terephthalaldehyde and p-tolualdehyde can be produced from p-xylene, phthalaldehyde and o-tolualdehyde from o-xylene, isophthalaldehyde and m-tolualdehyde from m-xylene, 2-methylterephthalaldehyde, 2,4-dimethylbenzaldehyde, 2,5-dimethylbenzaldehyde and 3,4-dimethylbenzaldehyde from pseudocumene, 3,5-dimethylbenzaldehyde, 5-methylisophthalaldehyde and 1,3,5-triformylbenzene from mesitylene, 2,5-dimethylterephthalaldehyde, 4,5-dimethylphthalaldehyde, 2,4,5-trimethylbenzaldehyde, 2,4,5-triformyltoluene and 1,2,4,5-tetraformylbenzene from durene, and so forth. Among them, the catalyst of the present invention is particularly suitable for producing terephthalaldehyde from p-xylene.

The catalyst of the present invention for partial oxidation of methylbenzenes can be represented by the following formula 1:
WOₓ (1)

where W stands for a tungsten atom, O stands for an oxygen atom and x is a number determined by the oxidation state of W, preferably in the range of 2 to 3.

The catalyst of the present invention can be supported on a fire-resistant inorganic support in order to improve activity, selectivity or physical durability. Typical examples of such fire-resistant inorganic support are α-alumina, silica, titania, zirconia, silicon carbide, etc.

In case the catalytic active component is supported on a fire-resistant inorganic support, the content of the support plus the catalytic active component is at least 5 wt %, preferably at least 12 wt % and more preferably at least 15 wt %, considering the object of the present invention. If the content is below 5 wt %, wanted reaction activity and terephthalaldehyde selectivity cannot be attained.

The supporting amount may depend on the pore volume of the support. A support with larger pore volume is advantageous in that the supporting amount can be increased.

According to the experiments performed by the inventors, conversion rate is improved but selectivity decreases as the surface area of the support increases. Based on several experiments, a support having a surface area of 0.5 m²/g or smaller, preferably 0.1 m²/g or smaller, and more preferably in the range of 0.005 m²/g to 0.05 m²/g, is advantageous in terms of methylbenzene conversion rate and terephthalaldehyde selectivity, as complete oxidation of methylbenzenes and side reactions can be prevented. Within this range, the conversion rate increases as the surface area increases.

Further, a support having an average pore size of at least 10 μm, preferably at least 50 μm, is advantageous in terms of terephthalaldehyde selectivity.

The catalyst of the present invention for may be prepared by any conventional catalyst preparation method, without specific limitation. Conventionally, a support is dipped in an ammonium metatungstate solution and dried by evaporating the solution. After drying at 80-200° C., the support is baked at 300-700° C. to obtain a catalyst. In case no fire-resistant inorganic support is used, the solution is dried by evaporation, dried at the same temperature as above, crushed and processed, and then baked at the same temperature as above to prepare a catalyst.

The tungsten source used in preparing the catalyst is not particularly limited. In addition to the ammonium salt, an oxide, a carbide, a chloride, a sulfide, a silicide, an organic acid salt, a heteropoly acid, etc. can be used.

The solvent used to prepare a homogeneous solution or suspension is not particularly limited, either. For the solvent, water and alcohols such as methanol, ethanol, propanol and diol can be used. Preferably, water is used in terms of environmental protection. The water includes distilled water and deionized water.

Tungsten content of the solution or suspension is not particularly limited, but a high concentration is preferable in order to reduce catalyst preparation time. And, aqueous solution is preferable to suspension in view of catalyst uniformity.

Methods of drying the catalyst and supporting on the fire-resistant inorganic support are not particularly limited. Supporting can be performed by precipitation, impregnation, coprecipitation or coating. Among them, impregnation is preferable, because preparation of uniform catalyst and control of supporting amount are facile.

Method or atmosphere for drying or baking the catalyst is not particularly limited, either. To take non-limiting examples, vacuum drying, freeze drying, spray drying, microwave drying, rotary evaporation, air drying, etc. can be performed. And, drying or baking can be performed under air atmosphere, high oxygen atmosphere, low oxygen atmosphere, reductive atmosphere or inert gas atmosphere or in vacuum.

Shape of the catalyst or type of the fire-resistant inorganic support is not particularly limited. Any shape, including sphere, pellet, ring and honeycomb, is possible and any form, including oxide or hydroxide particle, gel and sol, is allowed.

The present invention also provides a method for producing aromatic aldehydes from partial oxidation of methylbenzenes in gas phase using molecular oxygen using the afore-mentioned catalyst. The methylbenzene used as source material of the partial oxidation according to the present invention is not particularly limited. Preferably, it is a methylbenzene having 8 to 10 carbon atoms. The present invention is particularly adjustable for producing terephthalaldehyde from p-xylene.

Besides methylbenzene and molecular oxygen, a diluent gas may be used, if required. And, air or pure oxygen may be used as source of the molecular oxygen. In general, the molecular oxygen is used in 3-100 moles per 1 mole of the methylbenzene. For the diluent gas, an inert gas such as nitrogen, helium, argon, etc., carbon dioxide, water vapor, etc. can be used.

Reaction condition of the oxidation of the methylbenzene in gas phase is not particularly limited. The reaction is performed by contacting the source gas with the catalyst at a space velocity of 1,000-100,000 hr⁻¹, preferably 1,000-50,000 hr⁻¹, and a reaction temperature of 350-700° C., preferably 450-650° C. The reaction is generally performed at normal pressure or at a slightly elevated pressure. However, it can be performed at a high pressure or at a reduced pressure. The reaction system is not particularly limited, either. For example, one-pass system or recycling system is possible and the reaction can be performed in a fixed bed, mobile bed or fluidized bed.

### BRIEF DESCRIPTION OF THE DRAWINGS

FIG. 1 shows p-xylene conversion rate versus TPAL selectivity for the catalysts of Example 1 and Comparative Examples 1 and 2.

FIG. 2 shows p-xylene conversion rate versus TPAL selectivity for the catalysts of Examples 1-3.

FIG. 3 shows p-xylene conversion rate versus TPAL selectivity for the catalysts of Examples 3-5.

### BEST MODES FOR CARRYING OUT THE INVENTION

Hereinafter, the present invention is described in further detail through examples. However, the following examples are only for the understanding of the invention and the invention is not limited to or by them.

Conversion rate, selectivity and one-pass yield are defined as follows.
Conversion rate (mol %)=(Moles of reacted materials/Moles of fed materials)×100;
Selectivity (mol %)=(Moles of each product/Moles of reacted materials)(Number of carbon atoms of each product/Number of carbon atoms of fed materials)×100;
One-pass yield (mol %)=(Moles of each product/Moles of fed materials)(Number of carbon atoms of each product/Number of carbon atoms of fed materials)×100

### TESTING EXAMPLE 1

### Comparison of Catalyst of the Present Invention with Conventional Catalyst for Partial Oxidation of Methylbenzenes

### EXAMPLE 1

An aqueous ammonium metatungstate solution was prepared to a concentration of 2 mmol/g as a tungsten source. 12.0 g of this solution was diluted with 60 mL of water. To the resultant solution was added 60 g of an α-alumina support SA5218 (Norton; 3/16-inch; spherical; surface area=0.008 m²/g; pore size=75 μm) which had been pre-heated at 120° C. Evaporation drying was performed while stirring the solution. After drying at 120° C. for 18 hours, baking was performed under air atmosphere at 650° C. for 2 hours. The obtained catalyst had a composition of 6.4% WOₓ/SA5218.

60 g of the catalyst was filled in a common continuous flow reactor. Reaction was performed under the following condition.

Reaction pressure: normal pressure

Reactant gas composition (volume ratio): p-xylene/oxygen/nitrogen=0.25/6.25/93.5 (oxygen/p-xylene=25)

Reactant gas feed rate: 1.2 L/min

Space velocity (GHSV): 1500 hr⁻¹

Reaction temperature: 450, 500, 550, 580° C.

Reactions of other Examples and Comparative Examples were performed under the same condition, unless specified otherwise, with the space velocity and kind of support and supporting amount varying. Reaction results are given in Table 1 and FIG. 1.

### COMPARATIVE EXAMPLE 1

Comparative Example 1 was performed to confirm the effect of tungsten. Vanadium was used as main component of the catalyst for partial oxidation. An aqueous ammonium metavanadate solution was prepared to a concentration 0.25 mmol/g as vanadium source. A catalyst was prepared in the same manner of Testing Example 1 except for using 144 g of this solution. The prepared catalyst had a composition of 4.8 wt % VOx/SA5218. Reaction temperatures were 400, 430, 470 and 510° C. Reaction results are given in Table 1 and FIG. 1.

### COMPARATIVE EXAMPLE 2

Comparative Example 2 was performed to confirm superiority of mono-component tungsten over the conventional multi-component tungsten oxide. An antimony tartrate solution was prepared to a concentration of 0.5 mmol/g as antimony source. To a solution in which 150 g of L-tartaric acid was dissolved in 310 mL of water was added 36.5 g of antimony trioxide. Then, an antimony tartrate solution was prepared by heat reflux. 40.4 g of iron nitrate enneahydrate was dissolved in water, so that the total solution weighed 100 g, to prepare a 1 mmol/g iron nitrate solution. A catalyst was prepared in the same manner of Example 1, except for adding 2.4 g of the antimony tartrate solution and 1.8 g of the iron nitrate to 18.0 g of the ammonium metatungstate solution of Example 1. The prepared catalyst had a composition of 10.7 wt % W₁₂Sb_{0.4}Fe_{0.6}Ox/SA5218. Reaction results are given in Table 1 and FIG. 1.

TABLE 1

| | | | Selectivity | | One-pass yield | |
|---|---|---|---|---|---|---|
| | Reaction | Conversion rate | (mol %) | | (mol %) | |
| Classification | temperature (° C.) | (mol %) | TPAL | PTAL | TPAL | PTAL |
| Example 1 | 450 | 8.3 | 67.9 | 15.8 | 5.6 | 1.3 |
| | 500 | 16.6 | 68.6 | 15.2 | 11.4 | 2.5 |
| | 550 | 33.1 | 72.8 | 10.9 | 24.1 | 3.6 |
| | 580 | 45.9 | 72.1 | 8.7 | 33.1 | 4.0 |
| Comparative Example 1 | 400 | 35.0 | 16.7 | 39.2 | 5.8 | 13.7 |
| | 430 | 80.6 | 13.4 | 26.4 | 10.8 | 21.3 |
| | 470 | 98.0 | 4.0 | 7.0 | 3.9 | 6.9 |
| | 510 | 99.0 | 2.4 | 3.4 | 2.4 | 3.4 |
| Comparative Example 2 | 450 | 61.3 | 14.4 | 2.5 | 8.8 | 1.5 |
| | 500 | 83.9 | 33.4 | 3.4 | 28.0 | 2.9 |
| | 550 | 92.9 | 26.7 | 2.3 | 24.8 | 2.1 |
| | 580 | 96.9 | 18.0 | 1.5 | 17.4 | 1.5 |

TPAL: terephthalaldehyde,
PTAL: p-tolualdehyde

As seen in Table 1 and FIG. 1, when the catalysts of Comparative Example 1 or Comparative Example 2 were used, p-xylene conversion rate was high but TPAL selectivity was very low. Particularly, the selectivity decreased as the conversion rate increased, showing that TPAL cannot be produced effectively at high conversion rate. On the contrary, the catalyst of Example 1 showed higher TPAL selectivity compared with Comparative Examples 1 and 2. In addition, the selectivity increased as the conversion rate increased.

### TESTING EXAMPLE 2

### Comparison of Catalytic Activity Depending on Catalyst Supporting Amount

Next, change of p-xylene conversion rate and TPAL selectivity was observed while changing supporting amount of the catalyst of the present invention.

### EXAMPLE 2

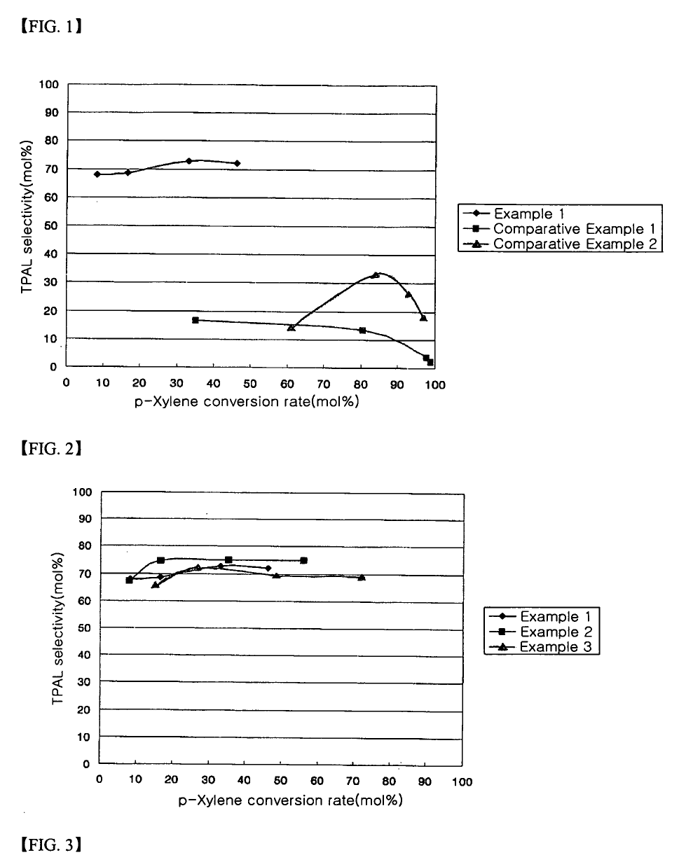

A catalyst was prepared in the same manner of Example 1 using 18.0 g of an aqueous ammonium metatungstate solution. A catalyst having a composition of 9.3 wt % WOₓ/SA5218 was obtained. Reaction results are given in Table 2 and FIG. 2.

### EXAMPLE 3

A catalyst prepared in the same manner of Example 1 using 36.0 g of an aqueous ammonium metatungstate solution. A catalyst having a composition of 17.8 wt %. WOₓ/SA5218 was obtained. Reaction results are given in Table 2 and FIG. 2.

TABLE 2

| | Reaction | Con- | | | | |
|---|---|---|---|---|---|---|
| | temper- | version | Selectivity | | One-pass yield | |
| Classi- | ature | rate | (mol %) | | (mol %) | |
| fication | (° C.) | (mol %) | TPAL | PTAL | TPAL | PTAL |
| Example 2 | 450 | 8.0 | 67.2 | 3.3 | 5.4 | 0.3 |
| | 500 | 16.4 | 74.8 | 3.6 | 12.3 | 0.6 |
| | 550 | 35.1 | 75.1 | 3.7 | 26.4 | 1.3 |
| | 580 | 55.9 | 75.1 | 3.7 | 42.0 | 2.1 |
| Example 3 | 450 | 15.3 | 65.7 | 4.2 | 10.1 | 0.6 |
| | 500 | 26.7 | 72.2 | 4.2 | 19.3 | 1.1 |
| | 550 | 48.5 | 69.7 | 4.5 | 33.8 | 2.2 |
| | 580 | 72.0 | 69.2 | 4.1 | 49.8 | 3.0 |

TPAL: terephthalaldehyde,
PTAL: p-tolualdehyde

As seen in Table 2 and FIG. 2, the catalytic activity increased as the supporting amount increased. Particularly, when the catalyst supporting amount was 17.8 wt % (Example 3), the conversion rate increased to 72%, which is much higher than Examples 1 and 2, in which the catalyst supporting amount was 6.4 wt % and 9.3 wt %, respectively. The selectivity was also very superior, in the range of 65% to 73%. Differently from conventional catalysts, TPAL selectivity did not vary a lot even at high conversion rate, which shows that TPAL can be produced effectively.

### TESTING EXAMPLE 3

### Comparison of Catalytic Activity Depending on Surface Area and Pore Size of Support

Change in catalytic activity of the catalyst of the present invention was observed while varying surface area and pore size of the support.

### EXAMPLE 4

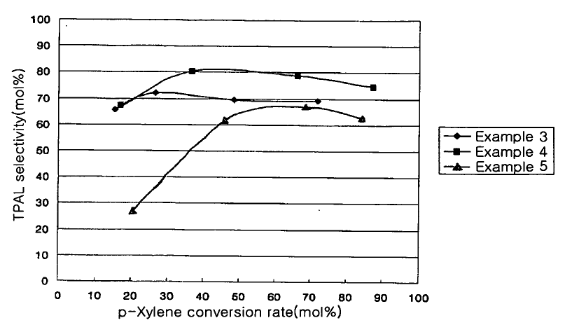

A catalyst was prepared in the same manner of Example 1 using an α-alumina support (SA5205; Norton; 3/16-inch; spherical; surface area=0.03 m²/g; pore size=130 μm) and 54 g of an aqueous ammonium metatungstate solution. A catalyst having a composition of 24.7 wt % WOₓ/SA5205 was obtained. Reaction results are given in Table 3 and FIG. 3.

### EXAMPLE 5

A catalyst was prepared in the same manner of Example 1 using a zirconia support (SZ5245; Norton; 3/16-inch; spherical; surface area=0.03 m²/g; pore size= 33/200 μm) and 54 g of an aqueous ammonium metatungstate solution. A catalyst having a composition of 22.9 wt % WOₓ/SZ5245 was obtained. Reaction results are given in Table 3 and FIG. 3.

TABLE 3

| | Reaction | Con- | | | | |
|---|---|---|---|---|---|---|
| | temper- | version | Selectivity | | One-pass yield | |
| Classi- | ature | rate | (mol %) | | (mol %) | |
| fication | (° C.) | (mol %) | TPAL | PTAL | TPAL | PTAL |
| Example 4 | 450 | 16.9 | 67.2 | 4.3 | 11.4 | 0.7 |
| | 500 | 36.7 | 80.3 | 3.7 | 29.5 | 1.4 |
| | 550 | 66.6 | 79.0 | 3.4 | 52.6 | 2.3 |
| | 580 | 87.4 | 74.8 | 3.3 | 65.4 | 2.9 |
| Example 5 | 450 | 20.5 | 27.2 | 4.2 | 5.6 | 0.9 |
| | 500 | 45.9 | 61.9 | 3.7 | 28.4 | 1.7 |
| | 550 | 68.5 | 67.1 | 3.2 | 46.0 | 2.2 |
| | 580 | 84.3 | 62.8 | 2.9 | 52.9 | 2.4 |

TPAL: terephthalaldehyde,
PTAL: p-tolualdehyde

As seen in Table 3 and FIG. 3, p-xylene conversion rate increased to 84% in Examples 4 and 5, in which the surface area of the support is larger than that of Example 3. Particularly, in Example 4, in which average pore size is larger, showed superior TPAL selectivity as well as high conversion rate.

### INDUSTRIAL APPLICABILITY

As described above, the catalyst for partial oxidation of methylbenzenes according to the present invention enables preparation of uniform catalyst compared with the conventional multi-component oxide catalyst.

Further, using the catalyst for partial oxidation of methylbenzenes according to the present invention, aromatic aldehydes can be produced from methylbenzenes with high selectivity and yield.

While the present invention has been described in detail with reference to the preferred embodiments, those skilled in the art will appreciate that various modifications and substitutions can be made thereto without departing from the spirit and scope of the invention as set forth in the appended claims.

1. A catalyst for partial oxidation of methylbenzenes comprising the compound represented by the following formula 1:
WOₓ (1)
where W stands for a tungsten atom, O stands for an oxygen atom and x is a number determined by the oxidation state of W.

2. The catalyst of claim 1, the compound represented by the formula 1 being supported on a fire-resistant inorganic support.

3. The catalyst of claim 2, content of WOₓ, or the active component, being at least 5 wt %.

4. The catalyst of claim 2, content of WOₓ, or the active component, being at least 12 wt %.

5. The catalyst of claim 2, surface area of the support being at most 0.5 m²/g.

6. The catalyst of claim 2, pore size of the support being at least 10 μm.

7. The catalyst of claim 1, the methylbenzene having 8 to 10 carbon atoms.

8. The catalyst of claim 1, the methylbenzene being p-xylene.

9. A method for producing aromatic aldehydes from partial oxidation of methylbenzenes in gas phase using molecular oxygen using the catalyst of claim 1.

10. The method of 9, the methylbenzene having 8 to 10 carbon atoms.

11. The method of 9, the methylbenzene being p-xylene and the produced aromatic aldehyde being terephthalaldehyde.
